(19) United States
(12) Patent Application Publication
(10) Pub. No.: US 2008/0006280 A1
(43) Pub. Date: Jan. 10, 2008

(54) Magnetic navigation maneuvering sheath

(76) Inventors: Anthony Aliberto, Laguna Hills, MN (US); Janothan C. Sell, Eagan, MN (US); Richard DiMonda, Marietta, GA (US); Michael Diaz, St. Louis, MO (US)

Correspondence Address: HARNESS, DICKEY, & PIERCE, P.L.C, 7700 BONHOMME, STE 400, ST. LOUIS, MO, 63105, US

(21) Appl. No.: 11/185,437

(22) Filed: Jul. 20, 2005

Related U.S. Application Data
(60) Provisional application No. 60/589,292, filed on Jul. 20, 2004.

(51) Int. Cl. A61B 19/00

(52) U.S. Cl. 128/899; 606/108

(57) ABSTRACT

The inventive apparatus may be slid onto a guiding catheter or other thru-lumen catheters, solid catheters, such as Electrophysiology catheters and multi-lumen tubing and advanced to the distal end of the guiding catheter or other thru-lumen catheters, solid catheters, such as Electrophysiology catheters and multi-lumen tubing to assist in holding the distal end of the guiding catheter or other thru-lumen catheters, solid catheters, such as Electrophysiology catheters and multi-lumen tubing at a target location within a patient, by utilizing an externally applied magnetic field to hold a plurality of magnetic elements on the apparatus in alignment with the target location. A medical device may then be advanced through the guiding catheter, for example, to the target location in the patient, without the guiding catheter backing out. The apparatus in combination with magnetic navigation thus provides support to hold the guiding catheter or other thru-lumen catheters, solid catheters, such as Electrophysiology catheters and multi-lumen tubing in place to prevent back out of the catheter.

### CROSS-REFERENCE TO RELATED APPLICATIONS

This application claims the benefit of U.S. Provisional Patent Application Ser. No. 60/589,292, filed Jul. 20, 2004. The disclosure of the above-referenced application is incorporated herein by reference.

### FIELD OF THE INVENTION

This invention relates to guide catheters, or other thru-lumen catheters, solid catheters, such as Electrophysiology catheters and multi-lumen tubing and more particularly to devices for use in guide catheters or other thru-lumen catheters, solid catheters, such as Electrophysiology catheters and multi-lumen tubing that may be magnetically steered within the body.

### BACKGROUND OF THE INVENTION

Guiding catheter devices, for example, have been used as a conduit for delivery of therapeutic tools into specific regions of the body, and may be manually guided by a physician to gain access to specific points in the vasculature system of a patient. For example, a guide catheter used in angioplasty may be inserted in the patient's arterial system through a puncture of the femoral artery, and a torque applied to the proximal end of the guide catheter to rotate the distal end while pushing the guide catheter. This action is repeated until, by trial and error, the guide catheter distal tip enters the desired vessel branch. Such trial and error methods can cause additional vessel wall contact in trying to reach the desired target location, possibly injuring the vessel. Some guide catheters have a pre-shaped end structure that aids in navigating the distal end of the catheter, and allows the mechanical pushing forces to be directed to the distal end of the catheter. However, physicians often experience back out of the guide catheter from the intra-arterial location, where the tip of the guide catheter moves away from its target location after being positioned there by the physician. Advancing guide wires or other medical devices through the guide catheter can also contribute to the back out of the guide catheter due to opposing forces, for example. Thus, there is a need for an apparatus that can position a guide catheter at a desired location in the vasculature of a patient, and for holding and anchoring the guide catheter in the desired location to resist back out.

### SUMMARY OF THE INVENTION

The present invention relates to an apparatus and method for navigating and positioning the distal end of a guiding catheter or other thru-lumen catheters, solid catheters, such as Electrophysiology catheters and multi-lumen tubing at a desired location within the vasculature of a patient, and for holding the guiding catheter or other thru-lumen catheters, solid catheters, such as Electrophysiology catheters and multi-lumen tubing to resist back out of the catheter from the desired location. In one embodiment, the apparatus comprises a flexible sheath having a proximal end and a distal end, a lumen therebetween, and one or more magnetically responsive elements disposed on the distal end of the sheath, whereby an externally applied magnetic field is used to preferentially align the one or more magnetic elements to guide the distal end of the sheath to a target location in the vasculature. In one preferred embodiment, the one or more magnetic elements are located around the flexible sheath near the distal end of the sheath. In a second preferred embodiment, a solid magnet is disposed within the end of the flexible sheath, and a side hole is provided near the distal end of the sheath through which the distal end of the guiding catheter or other thru-lumen catheters, solid catheters, such as Electrophysiology catheters and multi-lumen tubing may exit. In use, the distal end of the sheath, or the side hole, may be slid over the proximal end of the guiding catheter, or other thru-lumen catheters, solid catheters, such as Electrophysiology catheters and multi-lumen tubing and advanced along the guide catheter or other thru-lumen catheters, solid catheters, such as Electrophysiology catheters and multi-lumen tubing towards the distal tip. The sheath apparatus remains in place, and the guide catheter, for example, may be guided by the apparatus to the target location or vessel. Once positioned in a desired location, the applied magnetic field holds the magnetic element at the end of the sheath in alignment with the field to prevent back out of the guide catheter, or other thru-lumen catheters, solid catheters, such as Electrophysiology catheters and multi-lumen tubing. Guide wires and other medical devices are then able to be advanced through the inside of the guiding catheter, for example, to the desired vessel or target area without experiencing back out. The apparatus of the present invention in combination with a magnetic navigation system can thus hold the guide catheter and other thru-lumen catheters, solid catheters, such as Electrophysiology catheters and multi-lumen tubing in place to resist back out of the catheter.

According to one aspect of the invention, there is provided an apparatus for maintaining the placement of the distal end of a guide catheter or other thru-lumen catheters, solid catheters, such as Electrophysiology catheters and multi-lumen tubing at the target location within the vasculature of a patient, which method utilizes an externally applied magnetic field to align the magnetic element on the apparatus with the target location and hold or anchor the end of the apparatus in place. The magnetically navigable apparatus therefore provides stable placement of the distal end of the guide catheter or other thru-lumen catheters, solid catheters, such as Electrophysiology catheters and multi-lumen tubing at the desired target location.

Some embodiments of this invention provide an apparatus that can steer the distal end of a guide catheter or other thru-lumen catheters, solid catheters, such as Electrophysiology catheters and multi-lumen tubing towards a desired target, by the method of reorienting an externally applied magnetic field to deflect the distal end of the apparatus and realign the guiding catheter or other thru-lumen catheters, solid catheters, such as Electrophysiology catheters and multi-lumen tubing towards the desired target.

### BRIEF DESCRIPTION OF THE DRAWINGS

FIG. 1 is a side elevation view of the magnetically navigable maneuvering apparatus slid onto a guide catheter in accordance with the principles of the present invention;

FIG. 2 is an illustration of the magnetically navigable maneuvering apparatus slid onto a guide catheter and aligned with a target location, in accordance with the principles of the present invention; and

FIG. 3 is a second embodiment of a magnetically navigable maneuvering apparatus slid onto a guide catheter and aligned with a target location, in accordance with the principles of the present invention.

Correspondence reference numerals indicate corresponding parts throughout the several views of the drawings.

### DETAILED DESCRIPTION OF THE INVENTION

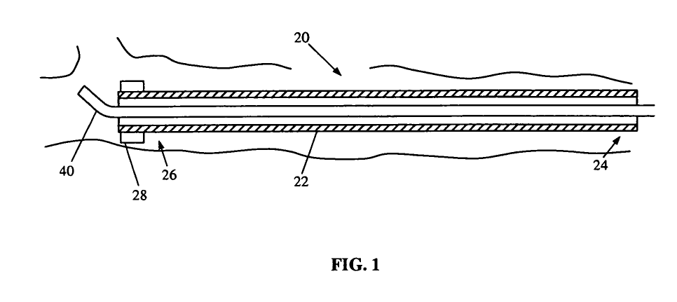

A preferred embodiment of an apparatus for magnetically establishing placement of the distal end of a guide catheter or other thru-lumen catheters, solid catheters, such as Electrophysiology catheters and multi-lumen tubing to a target location within a subject body in accordance with the principles of the present invention is indicated generally as 20 in FIG. 1. The apparatus 20 comprises a flexible sheath 22 having a proximal end 24 and a distal end 26, and a lumen therebetween. The flexible sheath 22 is preferably made of Pebax, Polyolefins, Polyurethane, Nylon, PET, Silicone, PTFE, polymer blends or other thermoplastic polymers but may alternatively be made from thermoplastic enhanced with braids and/or coils or other material providing suitable flexibility. The lumen through the sheath has inside diameter sufficient for accepting a typical guide catheter or other thru-lumen catheters, solid catheters, such as Electrophysiology catheters and multi-lumen tubing through the lumen of the sheath, and is preferably at least about 2.0 millimeters. The apparatus 20 further comprises one or more magnetically responsive elements 28 disposed around the sheath 22 near the distal end 26, wherein the magnetically responsive elements align with an externally applied magnetic field to align the distal end in a desired direction. The one or more magnetic elements 28 on the distal end 26 of the sheath 22 are held in alignment with the magnetic field to establish and maintain the position of the apparatus 20, which acts as an anchor to resist back out for the guiding catheter or other thru-lumen catheters, solid catheters, such as Electrophysiology catheters and multi-lumen tubing 30.

In a preferred embodiment, a magnetic element 28 is located at a minimum distance from the distal end 26 of the flexible sheath 22, and is preferably secured to the sheath 22 with an adhesive or other suitable bonding methods. The apparatus 20 may alternatively comprise a plurality of magnetically responsive elements 28 located adjacent to each other at a minimum distance from the distal tip of the sheath 22. The magnetically responsive elements 28 in the preferred embodiment preferably have a length of between about 2.0 and 20.0 millimeters, but may alternatively be any length or lengths that may suitably achieve the desired alignment. When the one or more magnetically responsive elements 28 at the distal end of the apparatus 20 are subjected to an externally applied magnetic field, the magnetically responsive elements 28 substantially align the distal tip with the direction of an externally applied magnetic field. The one or more magnetically responsive elements 28 can be made of a permanent magnetic material or a permeable magnetic material. The one or more magnetically responsive elements 28 can be made of such material and are of such dimensions that under the influence of an applied magnetic field of at least about 0.1 Tesla, and more preferably about 0.06 Tesla, the distal end portion of the apparatus substantially aligns with the local applied magnetic field direction. Suitable permanent magnetic materials include neodymium-iron-boron (Nd—Fe—B). Suitable permeable magnetic materials include magnetic stainless steel, such as a 303 or 304 stainless steel, Samarium Boron, Hiperco.

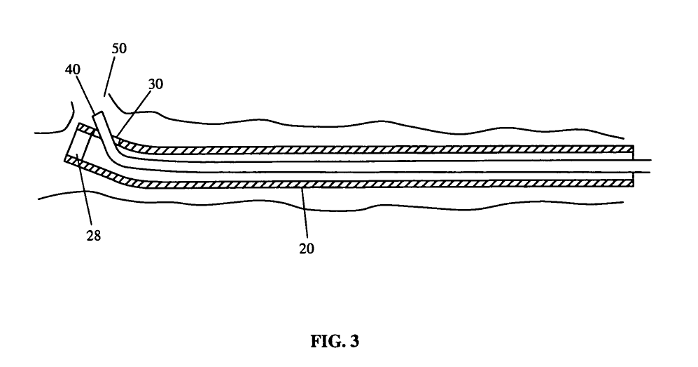

In an alternate embodiment, the apparatus 20 comprises a sheath 22 with a magnetically responsive element 28 disposed in the distal end 26, and a side hole 30 exiting the lumen through the sidewall of the sheath 22 as shown in FIG. 3. At the proximal end of the sheath is a hemostasis valve for allowing injection of saline or contrast, for example. The side hole 30 in the distal end of the apparatus 20 may be slid over the proximal end of the guiding catheter, or other thru-lumen catheters, solid catheters, such as Electrophysiology catheters and multi-lumen tubing after which the apparatus 20 may be advanced towards the distal end of the guide catheter 40 to aid in placement and support of the guide catheter 40 or other thru-lumen catheters, solid catheters, such as Electrophysiology catheters and multi-lumen tubing to prevent back out from the target location. The guide catheters, thru-lumen catheters, solid catheters, such as Electrophysiology catheters and multi-lumen tubing may alternately be advanced from the proximal end of the sheath.

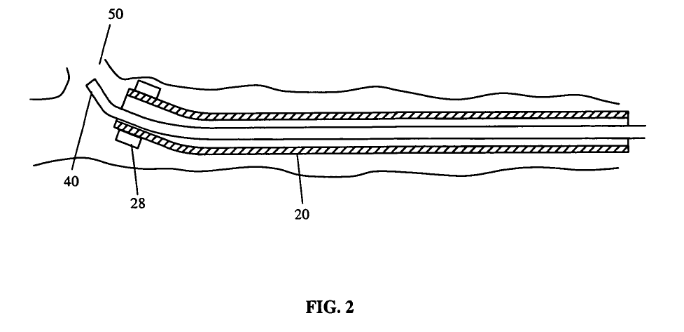

In operation, the apparatus 20 of the present invention is slid over the proximal end of a guiding catheter and advanced over the catheter into the subject body's vasculature. Once the distal end 26 of the apparatus 20 has been advanced to the distal end of the guiding catheter 40, a computer controlled magnetic navigation system may be used to apply a magnetic field to the region containing the distal tip of the apparatus 20 to orient the distal end in a desired direction. By controlling the direction of the applied magnetic field, the magnetically responsive elements 28 of the apparatus 20 may be aligned with the external magnetic field to orientate the distal tip 26 in a selected direction as shown in FIG. 2. The tip of the apparatus 26 is preferably capable of being deflected a minimum of 10 degrees relative to the longitudinal axis of the apparatus 20 when subjected to a magnetic field having a direction substantially perpendicular to the longitudinal axis of the apparatus 20 is a magnetic field as low as 0.1 Tesla, and more preferably a magnetic field as low as 0.06 Tesla. Once the distal end 26 has been aligned in the desired direction, the proximal end 24 of the apparatus 20 may then be pushed by hand to advance the distal end 26 though the subject body towards a target location such as the ostium 50 of a vessel for example, as shown in FIGS. 2 and 3. Alternatively, the proximal end can be pushed by an advancement mechanism under the manual control, or the control of a computer. The external magnetic field may be changed in orientation to realign or redirect the tip of the apparatus 20 in a stepwise process until the distal end of the guide catheter 40 or other thru-lumen catheters, solid catheters, such as Electrophysiology catheters and multi-lumen tubing has been steered toward the target location. The apparatus 20 remains in place, and the guide catheter 40 or other thru-lumen catheters, solid catheters, such as Electrophysiology catheters and multi-lumen tubing may then be advanced and positioned with the apparatus 20 in the ostium 50 of a target vessel or other target location. Guide wires and other medical devices are then able to travel through the inside of the guide catheter, for example, and out the end 40 or side hole to the desired vessel or target area. The magnetic field will continue to hold the magnetic elements 28 in alignment with the applied magnetic field direction, which will help in supporting the guide catheter or other thru-lumen catheters, solid catheters, such as Electrophysiology catheters and multi-lumen tubing in place while the physician advances guidewires or medical devices beyond the guiding catheter 40, for example, to the target location in the subject body. Accordingly, the apparatus 20 therefore facilitates access of the guiding catheter 40 or other thru-lumen catheters, solid catheters, such as Electrophysiology catheters and multi-lumen tubing to the target vessel, and also provides support for holding the guiding catheter 40 or other thru-lumen catheters, solid catheters, such as Electrophysiology catheters and multi-lumen tubing in place to prevent back out of the guiding catheter or other thru-lumen catheters, solid catheters, such as Electrophysiology catheters and multi-lumen tubing.

The advantages of the above described embodiment and improvements should be readily apparent to one skilled in the art, as to enabling placement and support of a guiding catheter or other thru-lumen catheters, solid catheters, such as Electrophysiology catheters and multi-lumen tubing in a target location. Additional design considerations may be incorporated without departing from the spirit and scope of the invention. Accordingly, it is not intended that the invention be limited by the particular embodiment or form described above, but by the appended claims.

What is claimed is:

1. An apparatus for positioning a guide catheter or other thru-lumen catheters, solid catheters, such as Electrophysiology catheters and multi-lumen tubing at a target location in the body, the apparatus comprising:
a flexible sheath having a proximal end and a distal end, and a lumen therebetween; and
one or more magnetic elements disposed on the distal end of the flexible sheath, wherein the distal end of the sheath is held in alignment with the target location through the interaction of an externally applied magnetic field associated with the one or more magnetic elements and at least one external magnetic field source outside the patient's body.

2. The apparatus of claim 1, wherein the lumen of the sheath has a minimum inside diameter sufficient for accepting a typical guide catheter, or other thru-lumen catheters, solid catheters, such as Electrophysiology catheters and multi-lumen tubing

3. The apparatus of claim 1, wherein the one or more magnetic elements are made of such material and are of such dimensions that under the influence of an applied magnetic field, the distal end portion of the apparatus substantially aligns with the local applied magnetic field.

4. The apparatus of claim 1, wherein the magnetic elements are made of a neodymium-iron boron.

5. The apparatus of claim 1, wherein the magnetic elements are made of a magnetic stainless steel.

6. The apparatus of claim 3, wherein the tip of the sheath is capable of being reoriented a minimum of 10 degrees relative to the axis of the distal end of the guide catheter, or other thru-lumen catheters, solid catheters, such as Electrophysiology catheters and multi-lumen tubing when subjected to a magnetic field having a reference angle 10 or more degrees relative to the axis of the distal end of the guide catheter or other thru-lumen catheters, solid catheters, such as Electrophysiology catheters and multi-lumen tubing.

7. The apparatus of claim 6, wherein the magnetically responsive elements respond to a field strength of as low as 0.1 Tesla.

8. The apparatus of claim 7 wherein the magnetically responsive elements respond to a field strength as low as 0.06 Tesla.

9. The apparatus of claim 7, wherein the one or more magnetic elements are secured to the sheath with an adhesive or other suitable bonding methods.

10. The apparatus of claim 2, wherein the inside diameter of the lumen is at least 2 millimeters.

11. An apparatus for positioning a guide catheter or other thru-lumen catheters, solid catheters, such as Electrophysiology catheters and multi-lumen tubing at a target location in the body, the apparatus comprising:
a flexible sheath having a proximal end and a distal end, a lumen therebetween, and a hole near the distal end providing an exit from the lumen through the side of the sheath; and
a magnetic element disposed in the distal end of the flexible sheath.

12. The apparatus of claim 11, wherein the magnetically responsive element in the distal end of the sheath is capable of orienting the distal end in a desired position at the target location through the interaction of an externally applied magnetic field associated with the magnetic element and at least one external magnetic field source outside the patient's body.

13. The apparatus of claim 12, wherein the lumen of the sheath has a minimum inside diameter sufficient for accepting a typical guide catheter or other thru-lumen catheters, solid catheters, such as Electrophysiology catheters and multi-lumen tubing

14. The apparatus of claim 13, wherein the magnetic element is made of such material and is of such dimensions that under the influence of an applied magnetic field, the distal end portion of the apparatus substantially aligns with the local applied magnetic field.

15. The apparatus of claim 14, wherein the magnetic element is made of a neodymium-iron boron.

16. The apparatus of claim 14, wherein the magnetic element is made of a magnetic stainless steel.

17. The apparatus of claim 14, wherein the tip of the sheath is capable of being reoriented a minimum of 10 degrees relative to the axis of the distal end of the guide catheter, when subjected to a magnetic field having a reference angle 10 or more degrees relative to the axis of the distal end of the guide catheter.

18. The apparatus of claim 15, wherein the magnetically responsive elements respond to a field strength of as low as 0.1 Tesla.

19. The apparatus of claim 18 wherein the magnetically responsive elements respond to a field strength as low as 0.06 Tesla.

20. The apparatus of claim 18, wherein the magnetic element is secured to the sheath with an adhesive or other suitable bonding methods.

21. The apparatus of claim 11, wherein the inside diameter of the sheath is at least about 2 millimeters.

22. The apparatus of claim 11, wherein the magnetic element has a length in the range of about 2 to about 30 millimeters.

23. An apparatus for positioning a guide catheter at a target location in the body, the apparatus comprising:
a flexible sheath having a proximal end and a distal end, a lumen therebetween, and a hole near the distal end exiting the lumen through the side of the sheath; and
a magnetic element disposed in the distal end of the flexible sheath, wherein the magnetic element in the distal end of the sheath is capable of orienting the distal end in a desired position at the target location through the interaction of an externally applied magnetic field associated with the magnetic element and at least one external magnetic field source outside the patient's body.

24. The apparatus of claim 23, wherein the lumen of the sheath has a minimum inside diameter sufficient for accepting a typical guide catheter, or other thru-lumen catheters, solid catheters, such as Electrophysiology catheters and multi-lumen tubing

25. The apparatus of claim 24, wherein the magnetic element is made of such material and is of such dimensions that under the influence of an applied magnetic field, the distal end portion of the apparatus substantially aligns with the local applied magnetic field.

26. The apparatus of claim 25, wherein the magnetic element is made of a neodymium-iron boron.

27. The apparatus of claim 25, wherein the magnetic element is made of a magnetic stainless steel.

28. The apparatus of claim 25, wherein the tip of the sheath is capable of being reoriented a minimum of 10 degrees relative to the axis of the distal end of the guide catheter, when subjected to a magnetic field having a reference angle 10 or more degrees relative to the axis of the distal end of the guide catheter, or other thru-lumen catheters, solid catheters, such as Electrophysiology catheters and multi-lumen tubing

29. The apparatus of claim 28, wherein the magnetic field is of a magnitude of about 0.1 Tesla.

30. The apparatus of claim 28 wherein the magnetically responsive elements respond to a field strength as low as 0.06 Tesla.

31. The apparatus of claim 27, wherein the magnetic element is secured to the sheath with an adhesive or other suitable bonding methods.

32. The apparatus of claim 25, wherein the inside diameter of the sheath is a minimum of 2 millimeters.

33. The apparatus of claim 25, wherein the magnetic element has a length in the range of about 2 to about 30 millimeters.

34. A method of supporting a guiding catheter or other thru-lumen catheters, solid catheters, such as Electrophysiology catheters and multi-lumen tubing using a sheath having one or more magnetically responsive elements near the distal end of the sheath, the method comprising the steps of:
inserting the distal end of the guiding catheter or other thru-lumen catheters, solid catheters, such as Electrophysiology catheters and multi-lumen tubing into the subject body and advancing the guiding catheter or other thru-lumen catheters, solid catheters, such as Electrophysiology catheters and multi-lumen tubing towards the target location within the subject body;
sliding the distal end of the sheath onto the proximal end of the guiding catheter or other thru-lumen catheters, solid catheters, such as Electrophysiology catheters and multi-lumen tubing and advancing the sheath in the subject body towards the distal end of the guiding catheter or other thru-lumen catheters, solid catheters, such as Electrophysiology catheters and multi-lumen tubing; and
subjecting the distal end of the sheath to a magnetic field applied external to the subject body so as to cause the one or more magnetically responsive elements to align with the direction of the magnetic field and hold the distal end of the guiding catheter or other thru-lumen catheters, solid catheters, such as Electrophysiology catheters and multi-lumen tubing in a desired direction and position.

35. The method of claim 34, further comprising the steps of:
reorienting the magnetic field to align the distal end of the sheath and guiding catheter or other thru-lumen catheters, solid catheters, such as Electrophysiology catheters and multi-lumen tubing with the desired target location; and
advancing the distal end of the guiding catheter or other thru-lumen catheters, solid catheters, such as Electrophysiology catheters and multi-lumen tubing to the target location in the subject body.

36. The method of claim 35, further comprising the step of advancing a medical device through the guiding catheter or other thru-lumen catheters, solid catheters, such as Electrophysiology catheters and multi-lumen tubing to the desired target location.
